(19) United States
(12) Patent Application Publication
(10) Pub. No.: US 2010/0059053 A1
(43) Pub. Date: Mar. 11, 2010

(54) APPARATUS CONFIGURED TO REDUCE MICROBIAL INFECTION AND METHOD OF MAKING THE SAME

(75) Inventor: William F. Niland, Stevensville, MD (US)

Correspondence Address: RATNERPRESTIA, P.O. BOX 980, VALLEY FORGE, PA, 19482, US

(73) Assignee: VAPOTHERM INC., STEVENSVILLE, MD (US)

(21) Appl. No.: 12/294,086

(22) PCT Filed: Mar. 22, 2007
(86) PCT No.: PCT/US07/07107

§ 371 Date: Feb. 10, 2009

Related U.S. Application Data
(60) Provisional application No. 60/784,979, filed on Mar. 23, 2006.
(60) Provisional application No. 60/785,231, filed on Mar. 23, 2006.
(60) Provisional application No. 60/784,980, filed on Mar. 23, 2006.

(51) Int. Cl. A61M 15/08

(52) U.S. Cl. 128/203.18

(57) ABSTRACT

A breathing gas delivery system is provided. The system includes a nasal cannula having a lumen and a nasal prong configured to deliver a gas from the lumen and into at least one nare of a patient through the nasal prong. At least one of the lumen and the nasal prong includes an amount of an antimicrobial agent effective to kill or inhibit growth of microorganisms on one or more surfaces of the nasal cannula. A method of delivering a breathing gas to a patient using the nasal cannula is also provided.

### BACKGROUND OF THE INVENTION

It has been recognized that the delivery of oxygen, oxygen-enriched air, and other breathing gases to the respiratory tract of a patient often results in discomfort to the patient, especially when the breathing gas is delivered over an extended period of time. It has also been recognized that the delivery of gases having relatively low absolute humidity can result in respiratory irritation.

Several devices have been proposed to overcome these problems. U.S. Pat. No. 4,632,677, issued to Richard H. Blackmer, the disclosure of which is incorporated herein by reference, describes an oxygen-enriching apparatus including means for increasing or regulating the humidity of the breathing gas supplied by the apparatus. The Blackmer apparatus employs an array of membrane cells, a vacuum pump to draw a flow of humidity-and-breathing gas from each cell, low- and high-temperature condensers connected to receive breathing gas drawn from the cells, and a proportioning valve connected to the condensers for providing a desired humidity level of the breathing gas.

Additionally, an exemplary system for delivering heated and humidified gas to a patient is described in application Ser. No. 10/149,356, filed Jan. 29, 2003, which is incorporated herein by reference in its entirety.

A nasal cannula is a minimally invasive apparatus for administering respiratory therapy to a patient via the nasal passageway. Treatment via a nasal cannula may require a patient to be in intimate and prolonged contact with the nasal cannula's outer surfaces. The outer surfaces of the cannula, when worn by a patient, contact the patient behind the ears, along the cheeks, along the upper lip, and within the patient's nares. After wearing a nasal cannula for an extended period, the often inevitable rubbing of its outer surface against the patient's skin due to patient movement, as well as possible patient perspiration, can cause irritation at these points of contact. Due to their weakened condition, these areas of irritated skin can present potential or perceived sites for local infection in some circumstances.

Treatment via a nasal cannula may also require a patient to be in intimate and prolonged contact with the breathing gases delivered through the nasal cannula. With respect to such breathing gas, should the gas stream delivered to the patient contain an appreciable microorganism content, such content may stress or be perceived to stress the upper and lower respiratory tract of the patient. It is worth noting that even where the design of the breathing gas source precludes or reduces the introduction of microorganisms, there may remain a perception among patients, hospital staff, and physicians with respect to possible infection.

There remains room for improvement in the field of breathing gas delivery.

### SUMMARY OF THE INVENTION

According to one exemplary embodiment, this invention provides a nasal cannula with an interior surface and an exterior surface, the nasal cannula having a lumen and a nasal prong configured to deliver a gas from the lumen and into at least one nare of a patient through the nasal prong. At least one of the lumen and the nasal prong includes an amount of an antimicrobial agent effective to kill or inhibit growth of microorganisms on at least one of the interior and exterior surfaces of the nasal cannula.

According to another embodiment of the invention, the nasal cannula includes tubing configured to receive a gas for delivery toward at least one nare of a patient; a lumen in fluid flow communication with the tubing; and a nasal element in fluid flow communication with the lumen, the nasal element including at least one nasal prong. An antimicrobial agent is incorporated into or applied to one or more of the tubing, the lumen, and the nasal element.

According to yet another embodiment of the invention, the nasal cannula includes a lumen configured to receive a breathing gas for delivery toward the nare of the patient, the lumen having an interior surface defining a passageway for the breathing gas. The lumen includes an amount of an antimicrobial agent effective to destroy or inhibit the growth of microorganisms within the passageway. The nasal cannula also includes a nasal prong in fluid flow communication with the lumen, the nasal prong being configured to be positioned within the nare of the patient for delivery of the breathing gas from the lumen and to the nare of the patient. The nasal prong has an exterior surface positioned for contact with an interior of the nare of the patient and includes an amount of an antimicrobial agent effective to kill or inhibit the growth of microorganisms on the exterior surface of the nasal prong.

According to yet another embodiment of the invention, a method is provided for delivering a breathing gas to a patient. The method includes positioning a nasal prong of a nasal cannula within a nare of the patient. Before or after such positioning, gas is delivered through a through a lumen of the nasal cannula. Gas is then directed toward the nasal prong of the nasal cannula to the patient. An effective amount of an antimicrobial agent is applied to or incorporated into the lumen or the nasal prong of the nasal cannula to inhibit the growth of microorganisms on one or more surfaces of the nasal prong or the lumen.

According to one exemplary embodiment, this invention provides a membrane configured to transfer water vapor to a breathing gas while inhibiting microbial growth. The membrane comprises a substrate having a water-contacting surface configured for contact with water and a gas-contacting surface configured for contact with gas. The substrate has porosity to deliver water vapor from the water-contacting surface to the gas-contacting surface. An amount of an antimicrobial component is provided in the substrate effective to inhibit microbial growth on at least one of the water-contacting surface and the gas-contacting surface.

Additionally, the present invention provides an apparatus adapted to transfer water vapor from water to a breathing gas while inhibiting microbial growth. The apparatus comprises a housing configured to receive water and a membrane positioned within said housing to separate the water from the breathing gas and configured to transfer water vapor from the water to the breathing gas. The membrane comprises a substrate having a water-contacting surface configured for contact with water and a gas-contacting surface configured for contact with gas. The substrate has porosity to deliver water vapor from water at the water-contacting surface to gas at the gas-contacting surface. An amount of an antimicrobial component is provided in the substrate effective to inhibit microbial growth on at least one of the water-contacting surface and the gas-contacting surface.

Further, the present invention provides a method of delivering humidified breathing gas to a patient while inhibiting microbial growth. The method comprises the steps of: transmitting water vapor to the breathing gas across a membrane comprising an amount of an antimicrobial component effective to inhibit microbial growth, thereby. humidifying the breathing gas; and delivering the humidified breathing gas to the patient.

Also, the present invention provides a method of delivering humidified breathing gas to a patient while inhibiting microbial growth. The method comprises the steps of delivering water vapor from water at a water-contacting surface of a substrate to gas at a gas-contacting surface of the substrate, thereby humidifying the gas; contacting water with an antimicrobial component associated with the substrate, thereby inhibiting microbial growth on at least one of the water-contacting surface and the gas-contacting surface of the substrate; and delivering the humidified gas to the patient.

The present invention also provides an apparatus adapted to transfer water vapor from water to a breathing gas. The apparatus comprises a housing configured to receive water and a membrane positioned within the housing to separate the water from the breathing gas and configured to transfer water vapor from the water to the breathing gas. The membrane comprises an amount of an antimicrobial component effective to maintain patency of the membrane.

Further, the present invention provides a method of maintaining patency of an apparatus adapted to transfer water vapor from water to a breathing gas and having a housing configured to receive water and a breathing gas and a membrane positioned within said housing to separate the water from the breathing gas and configured to transfer water vapor from the water to the breathing gas. The method comprising the steps of associating an antimicrobial component with a substrate to form the membrane; and positioning the membrane at least partially within the housing to at least partially define a space configured to contain water and a space configured to contain breathing gas.

The present invention also provides a method of maintaining patency of an apparatus adapted to transfer water vapor from water to a breathing gas and having a housing configured to receive water and a breathing gas and a membrane positioned within the housing to separate the water from the breathing gas and configured to to transfer water vapor from the water to the breathing gas. The method comprising the steps of transporting water from a water-contacting surface of the membrane to a gas-contacting surface of the membrane, thereby contacting water with an antimicrobial component associated with a substrate of the membrane; filtering microorganisms from the water to inhibit the passage of microorganisms to the gas-contacting surface of the membrane; and inhibiting the growth of microorganisms contacting the membrane, thereby maintaining patency of the membrane.

The present invention also provides a delivery tube adapted to deliver a heated and humidified breathing gas to a patient while transferring heat to the breathing gas and inhibiting microbial growth within the delivery tube. The delivery tube includes a first lumen for delivery of the heated and humidified breathing gas and a second lumen for circulating a heated fluid for transferring heat to the breathing gas in the first lumen. A partition has a fluid-contacting surface configured for contact with fluid in the second lumen and a gas-contacting surface configured for contact with gas in the first lumen. The partition separates the first lumen from the second lumen. An amount of an antimicrobial agent effective to inhibit microbial growth on at least one of the gas-contacting surface and the fluid-contacting surface is provided with the delivery tube.

Also, the present invention provides a method of making a delivery tube having antimicrobial properties. The method comprises the steps of: associating with a polymer an amount of antimicrobial agent effective to inhibit microbial growth in at least one of the first and second lumens; and forming the polymer into the delivery tube having a first lumen and a second lumen.

The present invention also provides a method of delivering a breathing gas to a patient while inhibiting microbial growth. The method comprises the step of connecting a delivery tube to a source of breathing gas, wherein the delivery tube comprises a first lumen for delivery of the heated and humidified breathing gas and a second lumen for circulating a heated fluid for transferring heat to the breathing gas in the first lumen. A partition has a fluid-contacting surface configured for contact with fluid and a gas-contacting surface configured for contact with gas. The partition separates the first lumen and the second lumen. An amount of an antimicrobial agent effective to inhibit microbial growth in at least one of the first and second lumens is provided with the delivery tube. The method further provides the step of delivering the breathing gas through the delivery tube to the patient.

Additionally, the present invention provides a system for delivering heated and humidified breathing gas to the nasal passageway of a patient. The system comprises a source of a breathing gas and a delivery tube coupled to receive the breathing gas from the source. The tube comprises a first lumen for the passage of the breathing gas, a second lumen for circulating a heated fluid for transferring heat to the breathing gas in the first lumen, and an amount of an antimicrobial agent effective to inhibit microbial growth in at least one of the first and second lumens.

Further, the present invention provides a method of delivering heated breathing gas to a patient while inhibiting microbial growth. The method comprises the steps of: delivering a breathing gas to a first lumen of a delivery tube; delivering a heating fluid to a second lumen of a delivery tube; heating the breathing gas with the heating fluid; contacting at least one of the breathing gas and the heating fluid with an antimicrobial component, thereby inhibiting microbial growth in the at least one of the breathing gas and the heating fluid; and delivering the heated breathing gas to the patient.

### BRIEF DESCRIPTION OF THE DRAWINGS

The foregoing summary, as well as the following detailed description of preferred embodiments of the invention, will be better understood when read in conjunction with the appended drawings, which are incorporated herein and constitute part of this specification. For the purposes of illustrating the invention, there are shown in the drawings embodiments that are presently preferred. It should be understood, however, that the invention is not limited to the precise arrangements and instrumentalities shown. In the drawings, the same reference numerals are employed for designating the same elements throughout the several figures. In the drawings:

FIG. 1 is an illustration of an embodiment of a nasal cannula according to aspects of this invention;

FIG. 2A is an illustration depicting an exemplary nasal cannula of the present invention worn by a patient;

FIG. 2B is an enlarged sectional view of portion 2B of FIG. 2A; and

FIG. 3 is an illustration of a system incorporating an exemplary nasal cannula of the present invention.

FIG. 4 is a schematic drawing of an exemplary embodiment of a system for delivering heated and humidified breathing gas to the nasal passageway of a patient;

FIG. 5 is a cross sectional view of a vapor transfer cartridge shown in the breathing gas delivery system of FIG. 4;

FIG. 6 is a cross sectional view of a hollow fiber membrane used in the vapor transfer cartridge of FIG. 5; and

FIG. 7 is a sectional view of a gas delivery tube according to an exemplary embodiment of the present invention.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention. The invention is best understood from the following detailed description when read in connection with the accompanying drawing figures, which show exemplary embodiments of the invention selected for illustrative purposes. The invention will be illustrated with reference to the figures. Such figures are intended to be illustrative rather than limiting and are included herewith to facilitate the explanation of the present invention.

Exemplary embodiments of a nasal cannula according to this invention have been discovered to help overcome disadvantages that may be associated with a conventional nasal cannula. More specifically, embodiments of a nasal cannula described below are configured for use in respiratory treatments that may require a patient to be in intimate and prolonged contact with a nasal cannula's outer surfaces and/or the breathing gases delivered through the nasal cannula.

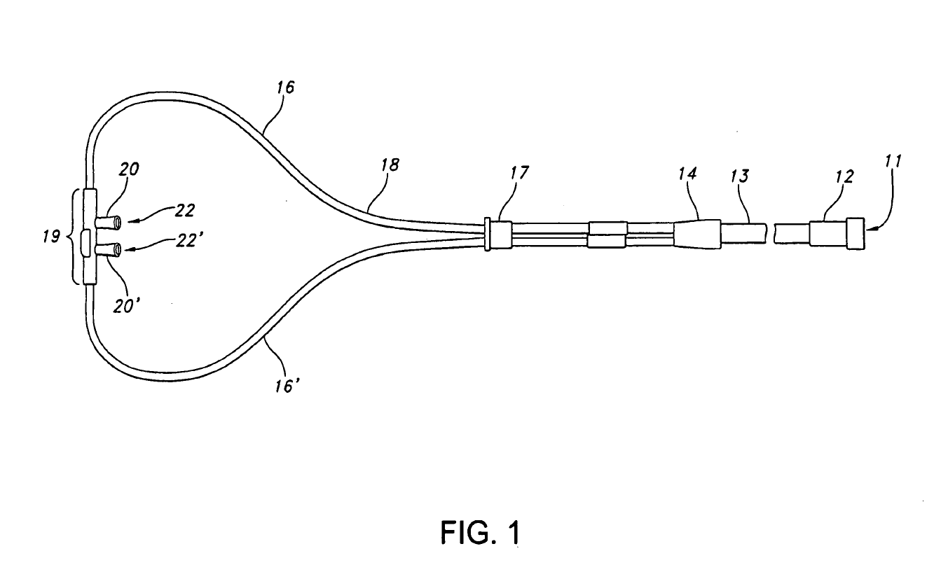

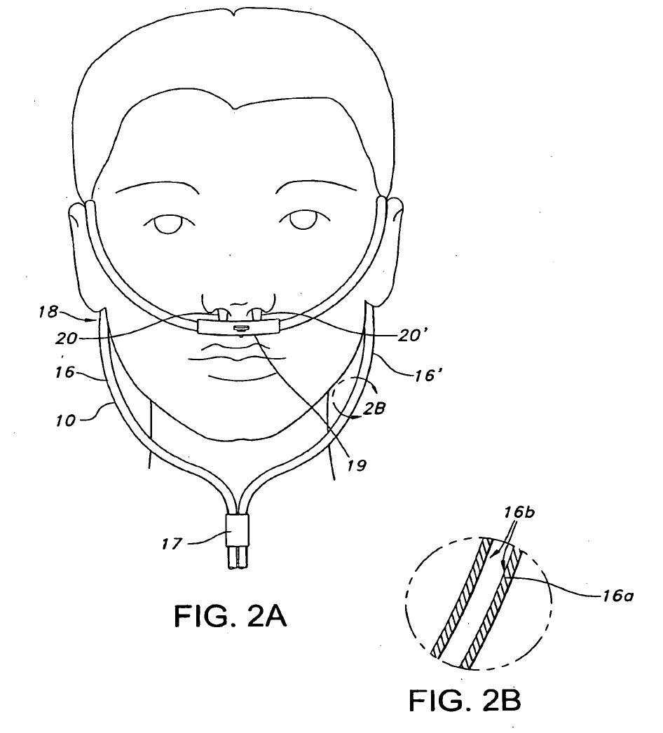

Referring to the embodiment illustrated in FIGS. 1 and 2A, a nasal cannula 10 according to a preferred embodiment of the present invention is shown. The nasal cannula 10 includes a connector fitting 12 at its distal end (farthest from patient), which fitting 12 defines a distal aperture 11. Connector fitting 12 is, coupled to tubing 13. At the proximal (closer to the patient) end of the tubing 13 are joined the first and second ends of lumens 16, 16′ at a juncture 14; thus forming a continuous loop 18. As illustrated in FIG. 2A, a nasal delivery element 19 is positioned on, and in fluid communication with, loop 18. Nasal delivery element 19 has two nasal prongs 20, 20′ defining respective proximal apertures 22, 22′ adapted for positioning in the patient's nares. Optionally, a slider 17 may be provided that surrounds both lumens 16,16′ and permits adjustment so that the nasal cannula 10 hangs more comfortably when worn by the patient.

At least one of the tubing 13, the lumens 16, 16′ and the nasal prongs 20, 20′ include an amount of an antimicrobial agent effective to kill or inhibit growth of microorganisms on one or more surfaces of the nasal cannula 10.

As shown in FIG. 2B, the lumen 16 of the nasal cannula 10 may have an interior surface 16B defining a passageway for the gas, and the lumen 16 optionally includes an amount of an antimicrobial agent effective to destroy or inhibit the growth of microorganisms on the interior surface 16B of the lumen 16. The lumen 16 also includes an exterior surface 16A, which optionally Includes an amount of an antimicrobial agent effective to destroy or inhibit the growth of microorganisms on the ID exterior surface 16A of the lumen 16. Lumen 16′ is the same as lumen 16.

The nasal prongs 20, 20′ of the nasal cannula 10 optionally include an amount of an antimicrobial agent effective to kill or inhibit the growth of microorganisms on an exterior surface of the nasal prongs 20, 20′. Microorganisms may include, but are not limited to, bacteria, fungi, viruses, algae, and protozoa.

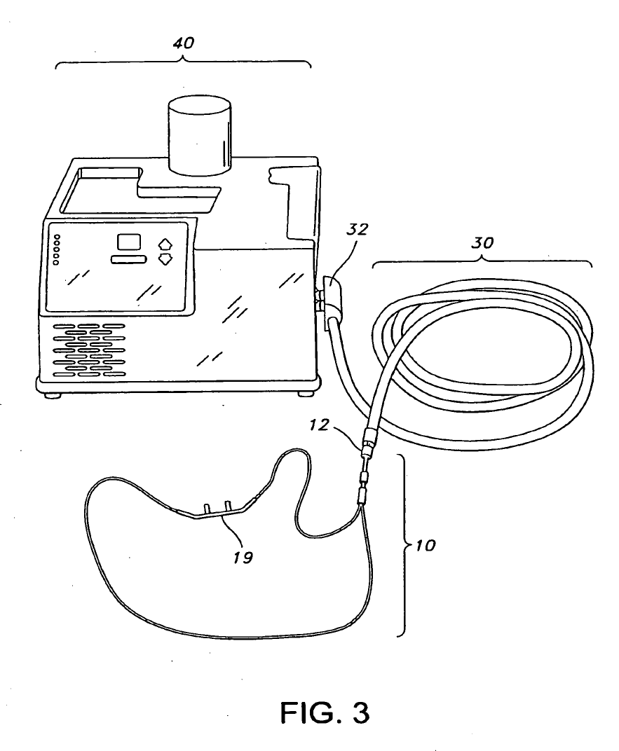

A system including nasal cannula 10 (generally illustrated in FIG. 3) is also provided. The system includes nasal cannula 10 coupled to a delivery tube 30 by connector fitting 12. A delivery tube 30 is coupled to source of breathing gas 40 by coupling 32. As shown, source 40 may be a stand-alone unit or a house supply such as is commonly found in a hospital or other clinical setting (not shown). Source 40 may optionally include humidification and/or heating capabilities so as to provide a heated and/or humidified breathing gas to the patient.

In use, nasal prongs 20, 20′ of the nasal cannula 10 are each positioned within a separate nare of the patient, as shown in FIG. 2A. Before or after such positioning, gas is delivered through lumens 16, 16′ of the nasal cannula 10 toward the nasal prongs 20, 20′ of the nasal cannula 10. Growth of microorganisms on one or more surfaces of the nasal prongs 20, 20′ or the lumens 16, 16′ is inhibited with an effective amount of the antimicrobial agent applied to or incorporated into the lumens 16, 16′ and/or the nasal prongs 20, 20′ of the nasal cannula 10. Growth of microorganisms is inhibited on the interior surface 16B of the lumens 16, 16′, the exterior surface 16A of the lumens 16, 16′, and/or on the exterior surface of the nasal prongs 20, 20′, and in an exemplary embodiment, all of these surfaces.

Applicants have discovered that the nasal cannula 10 produced according to aspects of this invention confers significant benefits. For example, by incorporating one or more antimicrobial agents into one or more subcomponents of the nasal cannula 10, microorganism growth on either or both of the inner and outer surfaces 16A, 16B of the nasal cannula 10 itself can be controlled or even eliminated. The Inhibition of microorganism growth, and the associated reduction of a risk of infection, is brought about at external points of contact between the nasal cannula 10 and the patient's skin such as, but not limited to, the areas behind the ears, along the cheeks, at the upper lip, and at and within the nares. Additionally, the inhibition of microorganism growth also helps in controlling the growth of any microorganism populations borne through the nasal cannula 10, thereby reducing the risk of a respiratory infection.

Such potential benefits become more apparent for patients receiving treatment via nasal cannula 10 that are in a generally weakened condition with reduced capacity to naturally ward off infection. By selecting nasal cannula components (e.g., the nasal delivery element 19, nasal prongs 20, 20′, lumens 16, 16′, tube 13, etc.) for treatment with one or more antimicrobial agents, a nasal cannula 10 can be produced to provide each of the forgoing benefits or combinations thereof.

A wide variety of antimicrobial compounds are suitable for use in nasal cannula 10 according this invention. For example, silver, silver salts, colloids, and complexes thereof are suitable to reduce and to control Infection. Likewise, other metals, such as gold, zinc, copper and cesium, also possess antimicrobial properties, both alone and in combination with silver.

An exemplary nasal cannula according an aspect of this invention can incorporate antimicrobial agents into a polymeric coating which is then applied to the surface of the cannula. For example, an antimicrobial agent is optionally incorporated into a coating solution in the form of a solution or a suspension of particles of the antimicrobial agent in the manner disclosed in U.S. Pat. No. 6,716,895 to Terry, incorporated herein by reference. Other examples of potentially suitable antimicrobial coatings are disclosed In U.S. Pat. No. 6,436,422 to Trogolo, et al. as well as U.S. Pat. No. 4,677,143 to Laurin, both of which are also incorporated herein by reference.

An additional exemplary nasal cannula according an aspect of this invention incorporate one or more antimicrobial agents or compounds such as silver, silver salts, and other antimicrobials within the polymeric substrate material from which one or more of the components of the cannula are formed. An antimicrobial compound may be physically incorporated into the polymeric substrate in a variety of ways. For example, a liquid solution of a silver salt may be dipped, sprayed or brushed onto the solid polymer, for example, in pellet form, prior to formation of the polymeric article. Alternatively, a solid form of the silver salt may be mixed with a finely divided or liquefied polymeric resin, which resin is then molded into the cannula.

Other processes may be used to incorporate an antimicrobial into, apply an antimicrobial to, or otherwise associate an antimicrobial with a nasal cannula component. For example, U.S. Pat. No. 4,592,920 to Murtfeldt et al. discloses a comminuted metal having a particle size of 30 microns or less. U.S. Pat. No. 4,849,223 to Pratt et al. discloses solutions that contain high concentrations of polymer or monomer solids and are, thus, viscous. U.S. Pat. No. 5,019,096 to Fox, Jr. et al. incorporates a synergistic amount of chlorhexidine and a silver salt in a matrix-forming polymer. U.S. Pat. No. 4,677,143 to Laurin et al. incorporates an antimicrobial metal into a binder having a low dielectric constant to form a coating. U.S. Pat. No. 4,933,178 to Capelli discloses a polymer coating containing an antimicrobial metal salt of a sulfonylurea. U.S. Pat. No. 5,848,995 to Walder discloses the solid phase production of polymers containing AgCl as an antimicrobial agent. The foregoing patents are incorporated herein by reference.

An exemplary nasal cannula embodiment includes, in one or more subcomponents, ion-exchange capable zeolite particulate materials with bound antimicrobial metals (e.g., silver, gold, zinc, copper and cesium) such as those subcomponents disclosed by U.S. Pat. No. 4,911,898 to Hagiwara, et al., which is also incorporated herein by reference. The means by which the antimicrobial zeolite materials are incorporated into polymeric substrates may vary. Notably, U.S. Pat. No. 4,775,585, to Hagiwara, et al., incorporated herein by reference, teaches two methods of incorporating the antimicrobial zeolite materials into polymer substrates. In one process, zeolite particles already bearing the necessary bound antimicrobial metals are mixed into the polymer or mixture of polymers at any stage prior to forming an article. In a second process, zeolite particles yet to be “loaded” with antimicrobial metals are incorporated prior to forming an article. After the article is formed, it is treated with a solution of one or more desired antimicrobial metal salts to “load” the zeolite particles.

One or more antimicrobial agents are provided in the components of the nasal cannula 10, namely in the lumens 16, 16′, the nasal delivery element 19, and the tubing 13. Antimicrobial agents are also provided in the remaining components depicted in FIG. 1. An antimicrobial agent may be provided In fewer than all, or in just one, of the components. For example, an antimicrobial agent can be provided in the nasal element 19 or in the lumens 16, 16′ but not in other components of the nasal cannula 10.

Antimicrobial agents can be selected from known agents including, but not limited to, silver and silver-containing compounds, zinc and zinc-containing compounds, gold and gold-containing compounds, cesium and cesium-containing compounds, quaternary ammonium compounds, and halogenated aromatic nitriles. The antimicrobial agents are added or otherwise applied to or associated with the nasal cannula In sufficient quantity to kill microorganisms on one or more surfaces of the cannula or to Inhibit growth of such microorganisms. Suitable antimicrobial agent(s) sufficiently rugged to withstand manufacturing processes can be incorporated into the polymeric material prior to the cannula's formation. Antimicrobial agent(s) added after the cannula is formed are also contemplated, such as, for example, a surface film coating and/or impregnation step. Combinations of an incorporated, coated, and impregnated antimicrobial agent(s) are likewise contemplated to optimize the previously identified antimicrobial effects desired (inner and outer surface of the cannula itself, patient skin, and/or patients' respiratory tract).

In a particular embodiment, the one or more antimicrobial agents are selectively applied via one or more of an incorporation, coating, or impregnation step to individual components of the nasal cannula apparatus.

In another series of embodiments, the antimicrobial agent(s) may be applied to the nasal delivery element 19, lumens 16, 16′, tubing 13, or fitting 12 or any combination thereof. In one such embodiment, it may be advantageous to restrict application of an antimicrobial compound to nasal delivery element 19 where the patient can be expected to suffer some degree of Irritation, where it is more moist, and where the patient's more delicate skin resides at the opening to the nares. In another embodiment, it may prove more advantageous to focus the antimicrobial compounds into fitting 12 to address potential microorganism growth at moisture collecting regions where fitting 12 is coupled to supply line 30. In yet further embodiments, it may prove advantageous to focus the antimicrobial compounds into either the lumens 16, 16′ or tubing 13 because these components have larger Interior and exterior surface areas, permitting maximal exposure of delivered breathing gases to antimicrobial compounds. As noted above, the desire to select one or more of these features may prompt a mix of the treated subcomponents to form an inventive device as contemplated herein.

Oxidation of the antimicrobial compound may cause at least some discoloration of the nasal cannula 10 over time. In order to make such discoloration less noticeable to the patient, the material from which the nasal cannula 10 is constructed may be colored with a pigment. The pigment may be added while the material from which the nasal cannula 10 is constructed is in a liquid form, to provide pigmentation throughout the nasal cannula 10. While the pigment may be added to the entire nasal cannula 10, those skilled in the art will recognize that the pigment may be added to only part of the nasal cannula 10, such as only one or more of the nasal delivery element 19, lumens 16, 16′, tubing 13, or fitting 12. No particular coloration is required.

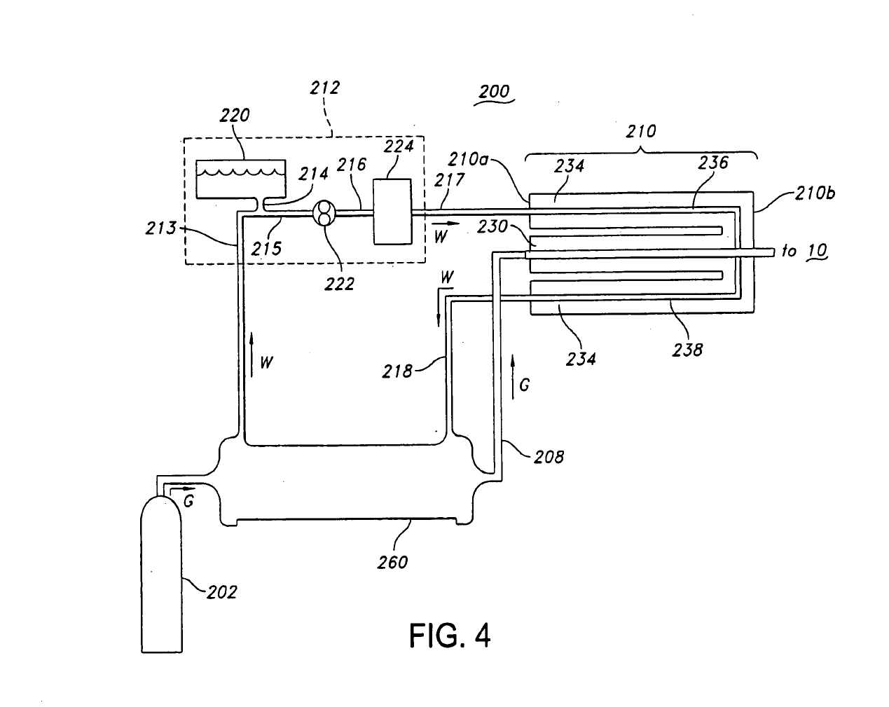

Referring to FIGS. 4-6, a vapor transfer cartridge and a gas delivery system incorporating the vapor transfer cartridge according to an exemplary embodiment of the present invention are disclosed. The vapor transfer cartridge includes an antimicrobial component that inhibits the growth of microorganisms within the vapor transfer cartridge and also within the fluids flowing through the vapor transfer cartridge.

FIG. 4 discloses a system 200 for delivering heated and humidified breathing gas to a patient. The system 200 includes a breathing gas supply 202 that supplies breathing gas to a vapor transfer cartridge 260. The vapor transfer cartridge 260 allows water to be transferred to the breathing gas to increase the humidity of the gas.

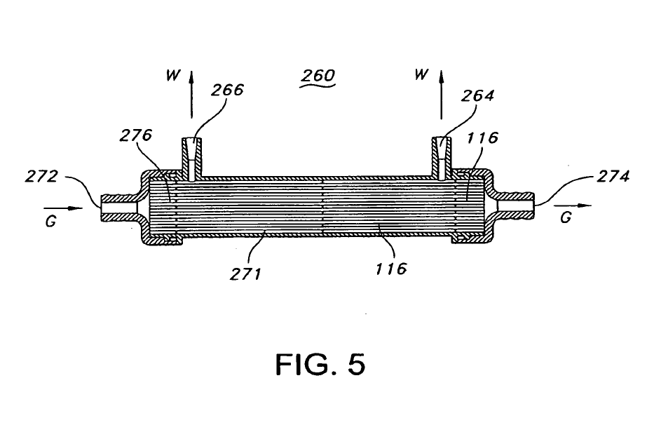

FIG. 5 illustrates an exemplary embodiment of the vapor transfer cartridge 260 for delivering water vapor to a gas. The cartridge 260 includes a housing 271 having a gas inlet 272 and a gas outlet 274. In an exemplary embodiment, the housing 260 may at least partially be formed from a polycarbonate material. Breathing gas enters the cartridge 260 at the gas inlet 272. A plurality of hollow fiber membranes 116 extend within the housing 271 between the gas inlet 272 and the gas outlet 274. The breathing gas travels from left to right as shown in FIG. 5 and exits the cartridge 260 at outlet 266.

Water enters the cartridge 260 at a water inlet 264 and contacts the outer surfaces of the hollow fiber membranes 116 with water. The water flows through the spaces between the outer surfaces of the hollow fiber membranes 116 and passes through pores in the walls of the hollow fiber membranes 116 to deliver water to the is flow of gas flowing through the cartridge 260. Water that is not transferred to the gas exits the cartridge 260 at water outlet 266 and is re-circulated to the fluid supply 220 for reuse.

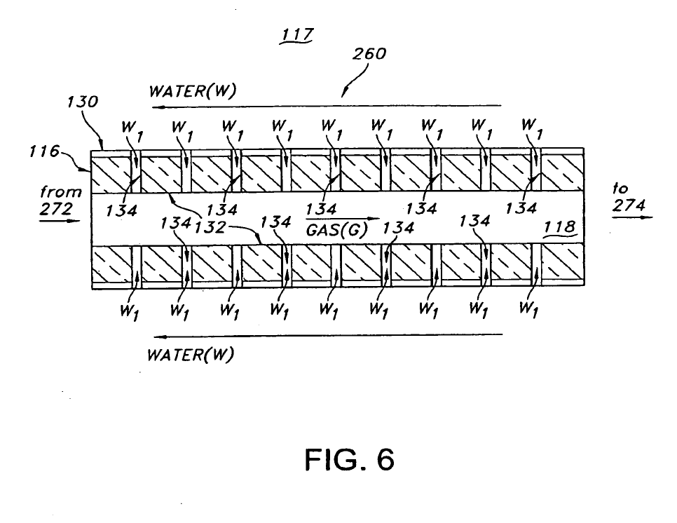

FIG. 6 illustrates the compartmental structure of the cartridge 260. Each membrane 116 includes a hollow fiber substrate 117, with each hollow fiber substrate 117 defining a passage 118 therein. In an exemplary embodiment, the fiber substrates 117 are constructed from a biocompatible polymeric material. Each fiber substrate 117 may be constructed from a material selected from the group consisting of cellulose acetate, polyvinylchloride, polyacrylonitrille, polycarbonate, polysulfone, polyamide, polyetherimide, polyimide, a combination thereof, or other suitable biocompatible materials.

The passages 118 provide for the flow of breathing gas from an upstream end of the passage 118 (from the gas inlet 272) to a downstream end of the passage 118 (to the gas outlet 274). Water supplied to the cartridge 260 flows over the substrates 117. Each substrate 117 includes a water-contacting surface 130 on the exterior of the substrate 117. A gas-contacting surface 132 defining the passage 118 opposes the water-contacting surface 130.

The water, shown by arrows “W”, contacts the water-contacting surface 130 as the water flows across the exterior of the membrane 116. Gas, shown by the arrow labeled “G”, contacts the gas-contacting surface 132 as the gas flows across the interior of the membrane 116. A plurality of pores 134 provide fluid communication between the water-contacting surface 130 and the gas-contacting surface 132. The pressure of the water supplied to the cartridge 260 is greater than the pressure of the gas supplied to the cartridge 260 so that the water is forced through the pores 134 and into the passages 118, as shown by the arrows “W₁”, where the water humidifies the gas flowing through the passages 118.

The pores 134 perform two functions. The pores 134 are large enough to allow water molecules to pass from the water-contacting surface 130 to the gas-contacting surface 132 in order to humidify the gas within the passage 118. Additionally, the pores 134 act as a filter to inhibit the passage of microorganisms from the water-contacting surface 130 to the gas-contacting surface 132.

The flow of gas G as it becomes humidified moves downstream to the end of the cartridge 260, where the breathing gas exits the cartridge 260 at the gas outlet 274. After the humidified gas exits the cartridge 260, the humidified gas travels through the gas delivery tube 210 and to the nasal cannula 10 for inhalation by the patient.

The cartridge 260 is configured to limit the transfer of water vapor to breathing gas to the point where little or no water is present in the liquid state in the breathing gas. According to an exemplary embodiment, no water is present in the liquid state in is the breathing gas, and the cartridge 260 is configured to maintain a relative humidity of about 100%.

The fiber membranes 116 of the cartridge 260 optionally include an amount of an antimicrobial agent effective to kill or inhibit the growth of microorganisms on a surface of the membrane 116. The antimicrobial agent may be on either or both of the water contacting surface 130 of the membrane 116 or the gas contacting surface 132 of the membrane 116.

An exemplary vapor transfer cartridge according an aspect of this invention can incorporate antimicrobial agents into a polymeric coating which may then applied to the surface of the membranes 116. The coating may be applied utilizing the same means as the nasal cannula 10 as described above.

Applicants have discovered that a vapor transfer cartridge produced according to aspects of this invention may confer significant benefits. For example, by incorporating one or more antimicrobial agents into one or more subcomponents of the vapor transfer cartridge, any microorganism growth on either or both of the inner and outer surfaces of the cartridge itself can be controlled or even eliminated. Additionally, the inhibition of microorganism growth also helps in controlling the growth of any microorganism populations borne through the vapor transfer cartridge, thereby reducing the risk of a respiratory infection.

Such potential benefits become more apparent for patients receiving breathing gas treatment who are in a generally weakened condition with reduced capacity to naturally ward off infection. By selecting a vapor transfer cartridge for treatment with one or more antimicrobial agents, a vapor transfer cartridge can be produced to provide each of the forgoing benefits or combinations thereof.

Additionally, growth of microorganisms on the membrane surfaces 130, 132 may block the pores 134 that allow transfer of water from the water-contacting surface 130 to the gas-contacting surface 132, thus reducing the ability of the cartridge 260 to humidify the breathing gas. The anti-microbial components inhibit the growth of, reduce, or eliminate microorganisms growing around the pores 134, thus maintaining patency of the membrane 116.

The system 200 also includes a fluid humidification and heating subsystem 212 that is used to both heat and humidify the breathing gas. The subsystem 212 includes a fluid supply 220 that supplies the fluid for humidification and heating of the breathing gas. In the exemplary embodiment shown, the fluid is water, although those skilled in the art will recognize that the fluid may be other fluids instead. Fluid is drawn from the fluid supply 220 by a pump 222. The pump 222 pumps the fluid from the fluid supply 220 to a heater 224, where the fluid is heated. In this embodiment, the heater 224 is an electrical heater, although the heater 224 may be another type of heater, such as a steam heater. The fluid supply 220, pump 222, and heater 224 are all in fluid communication with each other as well as the vap-or transfer cartridge 260 and the gas delivery tube 210 through tubing 213, 214, 215, 216, 217.

The heated fluid travels to the gas delivery tube 210, where the heated fluid heats the breathing gas in the gas delivery tube 210. After the fluid heats the breathing gas in the delivery tube 210, the fluid flows through tubing 218 to the vapor transfer cartridge 260, where the fluid humidifies the breathing gas. If the fluid is at a temperature higher than the breathing gas, the fluid also heats the breathing gas as well. Remaining fluid not transferred into the breathing gas flows back to the fluid supply 220, where the pump 222 recirculates the fluid. Make-up fluid is drawn from the fluid supply 220 to make up for water lost during the humidification of the breathing gas. The path of the fluid is generally a closed loop, meaning that the fluid recirculates through the system 200. However, it is noted that some fluid is lost from the system in the humidification process.

As shown in FIG. 4, the vapor transfer cartridge 260 utilizes a counter-flow of humidifying fluid relative to the breathing gas, meaning that the humidifying fluid is traveling in an opposite direction than the breathing gas. However, those skilled in the art will recognize that the humidifying fluid may travel in the same direction (parallel flow) as the breathing gas instead. Also, those skilled in the art will also recognize that the humidification of the breathing gas may alternatively be performed prior to heating the gas in the gas delivery tube 210.

Further, while the exemplary embodiment shown in FIG. 4 shows the heating of the breathing gas in the gas delivery tube 210 and the humidification of the breathing gas in the vapor transfer cartridge 260 to be in series, those skilled in the art will recognize that the heating and the humidification may be performed in parallel, or in two separate loops altogether.

The gas delivery tube 210 utilizes both parallel flow and counter-flow of the heating fluid to heat the breathing gas. As shown in FIG. 4, the heating fluid first begins heating the breathing gas in parallel flow when the breathing gas first enters the proximal end 230 of the gas delivery tube 210 from the vapor transfer cartridge 260. After the heating fluid travels the length of the gas delivery tube 210 from the proximal end 210a to the distal end 210b, the heating fluid is directed in a counter-flow direction to flow from the connection with the nasal cannula 10, where the heating fluid exits the proximal end 210a of the gas delivery tube 210 and travels toward the vapor transfer cartridge 260.

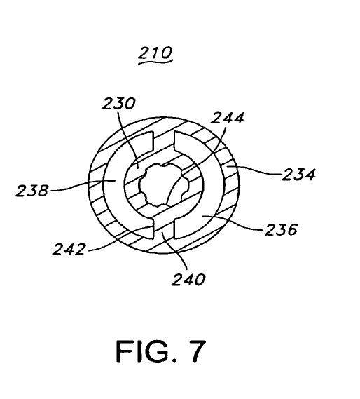

FIG. 7 shows a cross section of the gas delivery tube 210, showing a first lumen 230 that is co-axially disposed within the gas delivery tube 210 for delivering the breathing gas from the gas supply 202 to the nasal cannula 10. In the exemplary embodiment shown, the first lumen 230 has a generally “gear-shaped” cross section. However, those skilled in the art will recognize that the first lumen 230 may have other shapes, such as circular.

A second lumen 234 includes a supply portion 236 and a return portion 238. The supply portion 236 directs the heating fluid from the proximal end 210a of the gas delivery tube 210 to the distal end 210b of the delivery tube 210. The return portion 238 redirects the heating fluid from the distal end 210b of the delivery tube 210 to the proximal end 210a of the delivery tube 210. Each of the supply portion 236 and the return portion 238 has a generally “C-shaped” cross section, as can be seen in FIG. 7. The supply portion 236 and the return portion 238 together generally surround the first lumen 230.

A partition 240 within the gas delivery tube 210 separates the supply portion 236 from the return portion 238 and also defines the first lumen 230. The partition 240 also serves to separate the first lumen 230 and the second lumen 234 from each other. The partition 240 has a fluid-contacting surface 242 configured for contact with the heating fluid and a gas-contacting surface 244 configured for contact with the breathing gas.

An exemplary gas delivery tube 210 according an aspect of this invention can incorporate antimicrobial components into a polymeric coating which may then be applied to the surface of the gas delivery tube 210 defining the lumens 230, 234. In an exemplary embodiment, the gas delivery tube 210 is constructed from a biocompatible, flexible, polymeric material, such as vinyl. The tubing 204, 208, 213, 214, 215, 216, 217, 218 may be polymeric tubing from the same type or similar material to that of the gas delivery tube 210. An exemplary gas delivery tube 210 incorporates one or more antimicrobial components or agents such as silver, silver salts, and other antimicrobials within the polymeric material from which one or more of the components of the gas delivery tube 210 are formed. Optionally, the antimicrobial components may also be incorporated into the tubing 204, 208, 213, 214, 215, 216, 217, 218. The coating may be applied utilizing the same means as the nasal cannula 10 as described above.

Antimicrobial agent(s) added after the gas delivery tube 210 is formed are also contemplated, such as, for example, a surface film coating and/or impregnation step. Combinations of an incorporated, coated, and impregnated antimicrobial agent(s) are likewise contemplated to optimize the previously identified antimicrobial effects desired (inner and outer surface of the gas delivery tube itself and/or patients' respiratory tract). In an exemplary embodiment, the one or more antimicrobial agents are selectively applied via one or more of an incorporation, coating, or impregnation step to individual components of the gas delivery tube 210.

The antimicrobial agent is released into both/either of the breathing gas flowing through the first lumen 230 and/or the heating fluid flowing through the second lumen 234. The antimicrobial agent in the material forming the gas delivery tube 210 diffuses into the breathing gas to inhibit the growth of microorganisms in the breathing gas as the breathing gas is being inhaled by the patient.

The antimicrobial agent in the material forming the gas delivery tube 210 also diffuses into the heating fluid to inhibit the growth of microorganisms in the heating fluid. This capability is important since, after heating the breathing gas in the gas delivery tube 210, the heating fluid flows to the vapor transfer cartridge 260, where some of the heating fluid diffuses into the breathing gas, humidifying the breathing gas.

In addition to diffusing into both the heating fluid and the breathing gas to inhibit growth of microorganisms in the heating fluid and the breathing gas, the antimicrobial agent in the gas delivery tube 210 also inhibits the growth of microorganisms on the surfaces of the gas delivery tube 210 itself.

Applicants have discovered that gas delivery tube 210 produced according to aspects of this invention may confer significant benefits. For example, by incorporating one or more antimicrobial agents into one the gas delivery tube 210, microorganism growth on either or both of the inner and outer surfaces of the gas delivery tube 210 can be controlled or even eliminated. Additionally, the inhibition of microorganism growth also helps in controlling the growth of any microorganism populations borne through the gas delivery tube 210, thereby reducing the risk of a respiratory infection.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

1. A nasal cannula having an interior surface and an exterior surface, the nasal cannula including a lumen and a nasal prong configured to deliver a gas from said lumen and into at least one nare of a patient through said nasal prong, at least one of said lumen and said nasal prong comprising an amount of an antimicrobial agent effective to kill or inhibit growth of microorganisms on at least one of the interior and exterior surfaces of said nasal cannula.

2. The nasal cannula of claim 1 wherein said antimicrobial agent comprises silver.

3. The nasal cannula of claim 1 wherein said antimicrobial agent comprises an ion exchange carrier.

4. The nasal cannula of claim 1 wherein said antimicrobial agent is applied to a surface of said nasal cannula.

5. The nasal cannula of claim 1 wherein said antimicrobial agent is impregnated into said nasal cannula.

6. The nasal cannula of claim 1 formed from polymeric material.

7. The nasal cannula of claim 6 wherein said antimicrobial agent is incorporated into said polymeric material.

8. The nasal cannula of claim 1 further comprising tubing in fluid flow communication with said lumen and configured to deliver gas to the lumen.

9. The nasal cannula of claim 1, wherein the interior surface defines a passageway for the gas, said lumen comprising an amount of an antimicrobial agent effective to destroy or inhibit the growth of microorganisms within the passageway.

10. The nasal cannula of claim 1, said nasal prong being configured to be positioned within the nare of the patient, said nasal prong having an exterior surface positioned for contact with an interior of the nare of the patient, said nasal prong comprising an amount of an antimicrobial agent effective to kill or inhibit the growth of microorganisms on said exterior surface of said nasal prong.

11. A nasal cannula comprising:
a) tubing configured to receive a gas for delivery toward at least one nare of a patient;
b) a lumen in fluid flow communication with said tubing;
c) a nasal element in fluid flow communication with said lumen, said nasal element comprising at least one nasal prong;
wherein an antimicrobial agent is incorporated into or applied to at least one of said tubing, said lumen, and said nasal element.

12. The nasal cannula of claim 11 wherein said antimicrobial agent is applied to a surface of at least one of said lumen and said nasal element of said nasal cannula.

13. The nasal cannula of claim 11 wherein said antimicrobial agent is impregnated into at least one of said lumen and said nasal element of said nasal cannula.

14. The nasal cannula of claim 11, further comprising an interior surface defining a passageway for the gas, said interior surface comprising an amount of an antimicrobial agent effective to destroy or inhibit the growth of microorganisms within said passageway.

15. The nasal cannula of claim 11, said nasal prong of said nasal element being configured to be positioned within the nare of the patient, said nasal prong
having an exterior surface positioned for contact with an interior of the nare of the patient, said nasal prong comprising an amount of an antimicrobial agent effective to kill or inhibit the growth of microorganisms on said exterior surface of said nasal prong.

16. A nasal cannula configured to deliver breathing gas to at least one nare of a patient, said nasal cannula comprising:
a lumen configured to receive a breathing gas for delivery toward the nare of the patient, said lumen having a lumen interior surface defining a passageway for the breathing gas, said lumen comprising an amount of an antimicrobial agent effective to destroy or inhibit the growth of microorganisms within said passageway of said lumen; and
a nasal prong in fluid flow communication with said lumen, said nasal prong being configured to be positioned within the nare of the patient for delivery of the breathing gas from the lumen and to the nare of the patient, said nasal prong having an exterior surface positioned for contact with an interior of the nare of the patient and a nasal prong interior surface, said nasal prong comprising an amount of an antimicrobial agent effective to kill or inhibit the growth of microorganisms on at least one of said exterior surface and said interior surface of said nasal prong.

17. A method of delivering a breathing gas to a patient, said method comprising the steps of:
a) positioning a nasal prong of a nasal cannula within a nare of the patient;
b) delivering gas through a lumen of the nasal cannula toward the nasal prong of the nasal cannula; and
c) inhibiting growth of microorganisms on one or more surfaces of the nasal prong or the lumen with an effective amount of an antimicrobial agent applied to or incorporated into the lumen or the nasal prong of the nasal cannula.

18. The method of claim 17 wherein said inhibiting step comprising inhibiting the growth of microorganisms on an interior surface of the lumen.

19. The method of claim 17 further comprising the step of contacting an exterior surface of the nasal prong with an interior of the nare of the patient, said inhibiting step comprising inhibiting the growth of microorganisms on the exterior surface of the nasal prong.

20. A system for delivering heated and humidified air to the nasal passageway of a patient, the system comprising:
a) a source of a heated and humidified gas; and
b) a nasal cannula coupled to receive the gas from said source, said nasal cannula having a lumen and a nasal prong configured to deliver the gas from said lumen and into the nasal passageway of the patient through said nasal prong, at least one of said lumen and said nasal prong comprising an amount of an antimicrobial agent effective to kill or inhibit growth of microorganisms on one or more surfaces of said nasal cannula.

21-64. (canceled)
